(19) United States
(12) Patent Application Publication
(10) Pub. No.: US 2019/0002986 A1
(43) Pub. Date: Jan. 3, 2019

(54) Method to risk-stratify patients with cancer based on the comorbidities, and related differential gene expression information

(71) Applicants: Balaji Narayana Reddy, Boston, MA (US); Madanika Subhash, Boston, MA (US)

(72) Inventors: Balaji Narayana Reddy, Boston, MA (US); Madanika Subhash, Boston, MA (US)

(21) Appl. No.: 15/635,216

(22) Filed: Jun. 28, 2017

(51) Int. Cl. C12Q 1/68; C07K 14/475; C07K 14/82; C12P 19/34; G01N 33/574

(52) U.S. Cl. CPC C12Q 1/6886; C07K 14/475; A61K 38/00; C12P 19/34; G01N 33/57496; C07K 14/82

(57) ABSTRACT

A method to risk-stratify patients with cancer based on the comorbidities, and related differential gene expression is described here. This is of relevance when counseling and treating patients with various malignancies.

### SEQUENCE LISTING OR PROGRAM (IF APPLICABLE)

NA

### BACKGROUND

### Field of Invention

The present invention provides medical comorbidity, comorbidity of metabolic syndrome, genes related to any of these comorbidities, differential gene expression and gene sets the presence or expression of which is important in the diagnosis and/or prognosis of various cancer types, including progression to metastatic disease, and cancer related death.

### Prior Art

Medical comorbidities such as high blood pressure, diabetes, obesity, high cholesterol, smoking, alcohol consumption, are known to be associated with developing long-term illnesses such as heart disease, eye disease, kidney disease among others. Evidence also points to the risk of developing certain kinds of cancer such as kidney cancer. [1] Similarly, liver cancer, prostate cancer, thyroid cancer, pancreatic cancer, are among the types of cancer whose risk is similarly increased.

The National Cholesterol Education Program's Adult Treatment Panel III report (ATP III) defined criteria used to identify patients with the metabolic syndrome. ATP III identified six components of the metabolic syndrome: abdominal obesity, atherogenic dyslipidemia, raised blood pressure, insulin resistance with or without glucose intolerance, proinflammatory and prothrombotic states. When a subject has three of the five listed criteria, a diagnosis of the metabolic syndrome can be made.[2]

The initial diagnosis of cancer is based on a clinical suspicion. The tests conducted to confirm our suspicion of cancer are not always completely accurate. For example, a CT scan shows a kidney lesion, which is suspicious for cancer. The age and family history of a patient may point to a greater likelihood of cancer. In approximately 40% of the cases, these kidney lesions are non-cancerous.[3] In such clinical scenarios, additional tests such as a tissue biopsy, and/or genetic information may be of additional value, but its role is controversial.[4]

Patients with certain comorbidities such as metabolic syndrome, obesity, history of cigarette smoking, history of consuming alcohol, people of certain racial background, age, socioeconomic status, are known to have an increased risk of developing cancer. Similarly patients with other comorbidities known to be associated with increased risk of cancer include diabetes, high cholesterol, heart disease, and kidney disorder. Androgens also are thought to play an important role in cancer progression. Thus, patients harboring these comorbidities may have higher risk of harboring cancer despite an initial negative or an equivocal test or tests.

Following the diagnosis, physicians have a number of treatment options available to them including different combinations of no treatment, delayed treatment, surveillance, surgical treatment, chemotherapeutic drugs or a combination of treatments that are characterized as standard of care and a number of drugs or treatments that do not carry a label claim for a particular cancer but for which there is evidence of efficacy in that cancer. Best likelihood of good treatment outcomes requires that patients be assigned to optimal available cancer treatment and that this assignment be made as quickly as possible following diagnosis.

Cancer can present in various stages. An advanced stage cancer is usually worse in terms of severity, and/or survival than early stage cancer. Therefore, we rely on other predictors to identify the risk of having advanced disease, or identify those with greater risk of progressing to advanced disease. Identifying the patients who are less likely to progress is equally important. For example, an African American race is an important risk of worse cancer related outcomes in patients with prostate cancer. Similarly, drinking excessive alcohol is associated with worse outcomes in patients with liver cancer. Also, smoking is related to worse outcomes in lung and bladder cancer. Genetic factors also predict risk profile. For example, male gender is associated with worse bladder cancer outcomes. Patients with alterations in certain genes are associated with worse outcomes than those without. Breast cancer patients with BRCA-1 and BRCA-2 gene alterations have worse outcomes.

Currently, the clinical decisions do not always take into consideration the presence of comorbidities such as obesity, diabetes, high blood pressure, alcoholism, hormonal status, etc in prognosticating the patient's cancer related outcomes. The mutations as discovered using the “The Cancer Genome Atlas” (https://cancergenome.nih.gov) project, and similar experiments conducted in laboratories. One approach is to compile the top 5 or 20 or 100 or more genes (with changes in the genes or their expression levels) in a particular cancer type after conducting appropriate statistical analysis. In the same method, list of top 5 or 20 or 100 or more genes associated with worse outcomes are similarly compiled. One shortcoming of this approach is that, certain comorbidities, and gene alterations related to these comorbidities that may be driving the cancer disease is not taken into consideration.

Several recent studies have published in field of cancer diagnosis and prognostication based on gene expression analysis. Recently several groups have published studies concerning classification of various cancer types by micro array gene expression analysis. [5] Classification of certain tumor types based on gene expression pattern has also been reported. However, they do not provide the relationships of various comorbidities with the differentially expressed genes, and do not link the findings of treatment strategies in order to improve the clinical outcome of cancer therapy.

Given that there are approximately 20,000 genes in a human, and the number of genes that can attain statistically significant difference is quite large, it is difficult to achieve progress in developing effective strategies. Thus, it would become difficult to identify clinically relevant genes of interest in a large pool of statistically significant genes. Moreover, pursuing all of these genes and the gene products as potential diagnostic or therapeutic targets is impractical. We therefore narrowed our genes of interest to the genes associated with the comorbidities of interest. For example, high blood pressure is associated with development of various cancers. From the published literature, we quote an example of renal cell carcinoma that results as a result of either high blood pressure, or from being on medication to treat high blood pressure.[6] The risk of developing high blood pressure is often determined by the genetic make up of an individual, hormonal status, environmental factors that the patient is exposed to, etc. While the expression of these genes often is associated with high blood pressure, it may also be associated with other bodily functions, and disease processes. One such untoward outcome is cancer. By narrowing our focus to patients with medical conditions that lead to cancer, or medical conditions that lead to rapid progression of cancer, we can more effectively identify the genes (either alterations, or level of expression) associated with such medical conditions, and identify their role in cancer related outcomes. Furthermore, it provides an opportunity to explore diagnostic, therapeutic and prognostic applications by using the identified genes.

We describe a method to identify individuals at risk of developing certain cancers, progression of cancers, regression of cancer following therapy, progression of cancers leading to metastatic disease, and progression of cancers leading to death, based on certain gene alterations or the level of certain gene expression.

We describe a method to identify individuals at risk of developing certain cancer, progression of cancer, regression of cancer following therapy, progression to metastatic disease, and progression of cancer leading to death, based on the presence of factors leading to alterations in certain genes, leading to expression of these genes or presence of these gene products.

We describe a method to identify individuals at risk of developing certain cancers, progression of cancers, regression of cancer following therapy, progression of cancers leading to metastatic disease, and progression of cancers leading to death, based on the presence of certain gene alterations related to high blood pressure.

We describe a method that incorporates any drugs developed to block the expression of these genes or product of these genes alone or in combination with another chemotherapeutic agent or surgical therapy in preventing the progression of the disease.

We describe a method to detect the gene alterations, or their expression, which will help in identifying the risk of progression in individual patients.

This prognostic information may also be used to administer additional treatment or surgery with beneficial effect and outcome. This treatment may not always lead to a cure or a decrease in blood pressure, but may target other mechanism(s) to alter or inhibit the cancer growth.

### Prior Art

Some prior art we discovered:

U.S. Pat. No. 8,741,605 B2

US20150191792 A1

CA2934828 A1

US20150184247 A1

US201603265

### Previously Background*—Prior Art

Listed are the prior art.

In the studies so far, the identification of cancer genes, and identifying the role of cancer genes thus identified were by comparing normal controls to cancer patients, or, comparing normal tissue to cancer tissue, without consideration to the comorbidities of the patient.

None of the prior art discusses how to identify individuals at risk of developing certain cancers based on the presence of these comorbidities, or based on the presence of gene alterations and/or gene expression associated with these comorbidities, or based on the presence of factors leading to these gene alterations and/or gene expression.

None of the prior art discusses how to identify individuals at risk of faster progression of cancers based on the presence of these comorbidities, or based on the presence of gene alterations and/or gene expression associated with these comorbidities, or based on the presence of factors leading to these gene alterations and/or gene expression.

None of the prior art discusses how to identify individuals at risk of progression of cancers leading to metastatic disease based on the presence of these comorbidities, or based on the presence of gene alterations and/or gene expression associated with these comorbidities, or based on the presence of factors leading to these gene alterations and/or gene expression.

None of the prior art discusses how to identify individuals at risk of progression of cancers leading to death based on the presence of these comorbidities, or based on the presence of gene alterations and/or gene expression associated with these comorbidities, or based on the presence of factors leading to these gene alterations and/or gene expression.

The list of genes related to high blood pressure (hypertension), obesity and diabetes are ever increasing. One website http://bws.iis.sinica.edu.tw/THOD/ publishes the genes along with the related scientific articles related to these genes. In this website, searching for the term high blood pressure yielded a set of genes attached in file (1). Additional sources of hypertension genes include human-phenotype-ontology.github.io/. In this website, searching for the term high blood pressure yielded a set of genes attached in file (2). Other source include an article recently published, which provides a method to predict human hypertension genes.[7]

The understanding of the role of hypertension in cancer is well understood by knowing the pathophysiology of a cancer. Cancer grows by a method of new blood vessel formation, also called neovascularization. High blood pressure can also cause neovascularization leading to diseases such as hypertensive retinopathy. High blood pressure also induces changes in the blood vessels as a compensatory mechanism, and induces changes in almost all organs of the body. High blood pressure is also attributed to improper electrolyte metabolism by the kidneys. Renal cell carcinoma is also known to cause high blood pressure. The cause of high blood pressure is multifactorial. It is also likely due to interaction between multiple genoc.

### SUMMARY

Step 1: How did we identify the genes: 1) We looked up the list of high blood pressure genes published in various sources. Two such sources are: http://bws.iis.sinica.edu.tw/THOD/ and human-phenotype-ontology.github.io/. We use a statistical software to identify significant genes; 3) Top 5 or 10 or 20 genes; 4) Genes associated with high blood pressure, and closely linked to mTOR, PI3K, PTEN, and other known cancer genes; 5) While any of these methods described should not limit other ways to identify genes of interest in a particular patient, or a group of patients, as the genes attributed to causing high blood pressure is different in each individual, and so should the genes of high blood pressure related to causing cancer progression; 5) So, a highly expressed high blood pressure gene in an individual may be the target rather than a gene found to be most commonly expressed in patients with that particular caner. The genes of interest can be detected using microarray techniques known in the field.

How do we report the genes. The genes identified in a particular individual and the cancer risk profile may be generated into a report, so the patient may have this information. This report may also be detailed enough to provide necessary information to the treating physician.

In one embodiment, we selected the following set of genes from the list of high blood pressure genes: SCNN1B WNK1 WNK4 KCNJ5 CYP11B1 CYP11B2 PDE3A PRKG1 GUCY1A2. We then compared patients with stage 2 and lower cancer to stage 3 and higher cancer for difference in gene alterations, and gene expression.

These genes were picked from the list of high blood pressure genes. The gene names comply with the HUGO gene nomenclature committee guidelines.

In accordance with one embodiment, we used an online resource to explore the significance of these genes: http://www.cbioportal.org/. In this portal, we identified the subjects with renal cell carcinoma (clear cell type) of the TOGA, provisional data set comprising of 538 samples. We queried this website for the alterations in the said genes. We identified alterations in the genes as noted in FIG. (1a). We also noted that the cancer specific survival was significantly worse for subjects with alterations in the said genes. (FIG. 2a).

In accordance with one embodiment, we used an online resource: http://www.cbioportal.org/ to explore the significance of these genes. We identified the subjects with prostate adenocarcinoma of the TOGA, provisional data set comprising of 499 samples. We queried this website kir the mutations, in the said genes. We identified alterations in the genes as noted in FIG. (1b). We also noted that the cancer specific survival was significantly worse for subjects with mutations in the said genes. (FIG. 2b).

In accordance with one embodiment, we used an online resource: https://genome-cancer.ucsc.edu/proj/site/hgHeatmap/ to explore the significance of these genes.

We identified the subjects with renal cell carcinoma (clear cell type)of the TOGA, provisional data set comprising of 538 samples. We queried this website for the mutations, in the said genes. We then checked them for a statistically significant difference between patients with low (stage pT2 and lower) and high (stage pT3 and higher) stage renal cell carcinoma (clear cell type). (FIG. 3)

In one clinical scenario, a patient with Renal cell carcinoma, presented to the physician to be evaluated for the risk of his/her progression. Having identified the genes associated with worse prognosis in step 1, we used RT-PCR platform to identify the gene transcripts of the high blood pressure genes in the given patient. These genes may also be combined into a microarray as known in the field, to facilitate assessment of the patient sample for the gene alterations or gene expressions of interest. Some other techniques known in the field to identify the gene alterations include whole exome sequencing, and other gene sequencing technologies. These techniques of identifying the set of gene alterations, or gene expression in a patient are prior knowledge, and can be used effectively to identify the gene alterations or gene expression in a given patient. The test is performed on the biopsy of the cancer tissue, but could also be performed on the organ(s) harboring the cancer, blood, or other body fluids, circulating tumor cells, or stored tissue from the patient. The test could be performed serially in time to assess the changes in the genes of interest over time. The test sample if necessary is collected and stored in tubes that stabilize and prevent degradation of nucleotides or proteins of interest. The gene expressions are normalized against the expression levels of all RNA transcripts or their expression products in the tumor being evaluated, or a reference set of RNA transcripts or their products. If the gene alterations or the gene transcripts identified are among the genes associated with high risk for progression as identified in step 1, the patient can then be appropriately counseled on the appropriate treatment. Further, the treatment could be blood pressure lowering agents, agents that block the mutated gene(s) in that patient, or block the products of the gene(s). Further, serial measurement of the alterations in the gene or gene products could provide information related to the progression of the disease.

Similar method can be used to identify individuals potentially at higher risk of harboring high risk RCC. Additionally, potential treatments include blood pressure lowering agents and agents that block the by products of these genes, which can play a role in halting, reversing, or limiting the progression of the cancer. This is not limited to clear cell type renal cell carcinoma, or prostate cancer, and can be extrapolated to other tumor types as well. This is not limited to two groups of stage 2 and lower compared to stage 3 and higher. The comparison groups may include stage 1 to stage 2 and higher; stage 3 and lower compared to stage 4 and higher; or between any tumor classification types or between any tumor groups comparing lower to higher risk groups, as long as there is a statistically significant difference between the groups can be demonstrated. The difference in the genes can be used to identify individuals potentially at higher risk of harboring high risk RCC. Additionally, potential treatments include blood pressure lowering agents and agents that block the by products of these genes, which can play a role in halting, reversing, or limiting the progression of the cancer.

In other embodiments,

1. We create a training set (⅔rd of the cohort), and a validation set (⅓rd of the cohort). This division of dataset may be done by randomly picking patients into each of the sets randomly.

2. In the training set, we identify statistically different DNA mutations, methylation, differential gene expression, RNA and protein expression of genes between two groups. The two groups could be different tumor stages, age (eg:>70 years versus <=70 years), smokers vs non-smokers, alcoholics versus non-alcoholics, gender (male versus female), hormonal status (normal versus abnormal hormone levels or response), tumor versus controls, metastatic versus non-metastatic disease, or any other parameters to assess gene alterations and their differential expression. The groups could be more than two. The genes of interest can also be modified. Eg: if a patient has a certain gene altered, we could look for that gene in this model. Other methods of identifying genes of interest include any other well-known statistical methods in the field. One such method is top 5, or 10,or 20 altered genes could also be assessed by this method.

3. In the validation set, we confirm if the findings of the training step are true.

The entire steps 1 to 3 may be automated to perform approximately 1000 times, to ensure validity (that is, the identified mutations, methylation, RNA and protein expression; and agreement between the training and validation sets). There are several ways to perform statistical methods, and any such common knowledge methods can be used to identify the genes of interest. The comorbidity, gene, or gene products can then be evaluated for potential therapeutic targets, thereby achieving either a cure, or a delay in progression of cancer. In one embodiment, a blood pressure medication called angiotensin receptor blocker may be used to control high blood pressure. The same drug may be used to reduce the incidence of kidney cancer, and/or progression of kidney cancer. The resources such as: http://www.reactome.org and http://www.genome.jp/kegg/pathway.html provide us with necessary tools to explore drug targets, the method of which is within the realm of one skilled in the art.

In another embodiment, the gene alterations identified by statistical analysis could be ranked based on their close association with known cancer genes. The statistical analysis may also include multivariate analysis, with one more of the patient baseline characteristics data, gene alteration data, gene expression data being included in such multivariate analysis. Other common methods of statistical analysis known in the art may also be used. The links for some of the software available in the field are:

https://bioconductor.org/packages/devel/bioc/vignettes/TCGAbiolinks/inst/doc/tcgaBiolin ks.html

http://www.bioconductor.org

### DRAWINGS—FIGURES

FIG. 1a. Gene alterations in subjects with renal cell carcinoma (clear cell type) of the TCGA, provisional data set comprising of 538 samples.

FIG. 2a. Cancer specific survival of subjects with renal cell carcinoma (clear cell type), with alterations in the said genes.

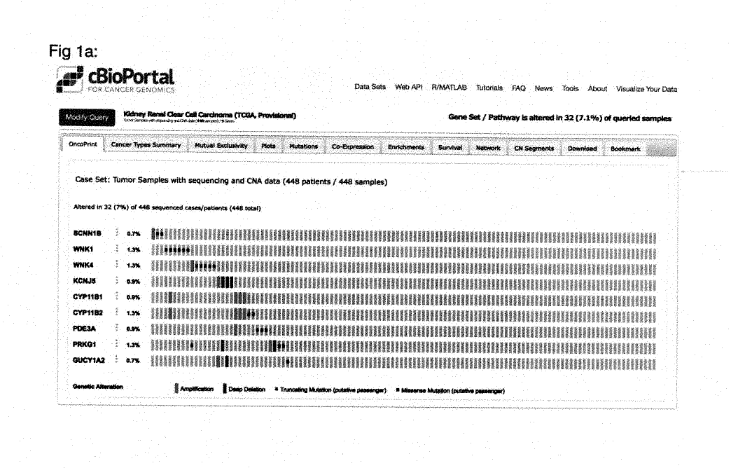

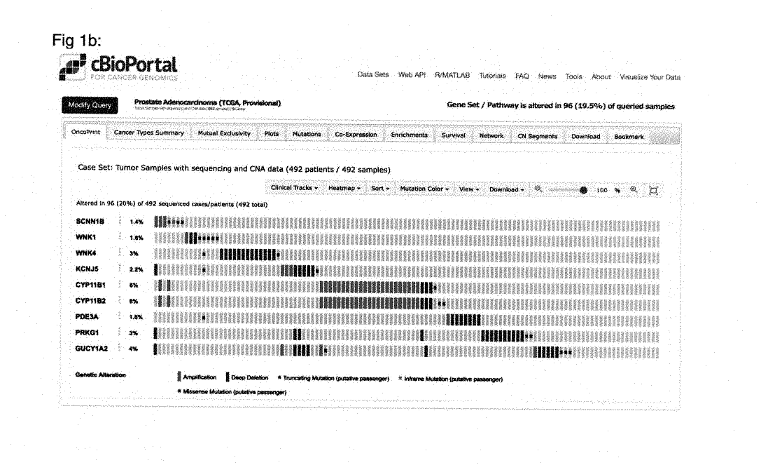

FIG. 1b. Gene alterations in subjects with prostate adenocarcinoma of the TCGA, provisional data set comprising of 499 samples.

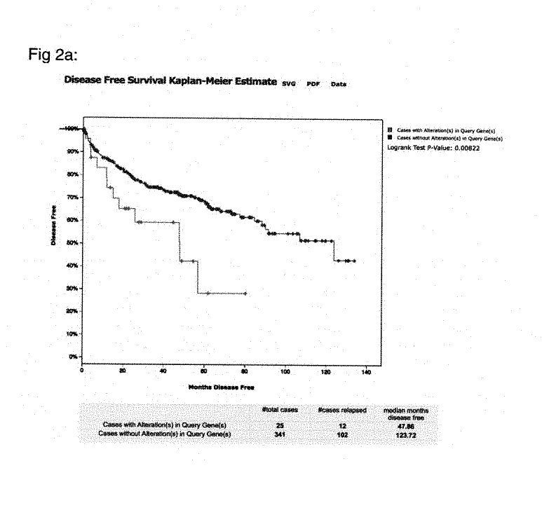

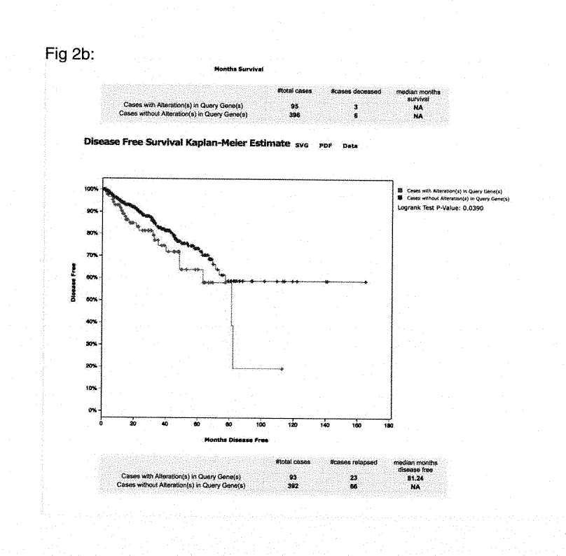

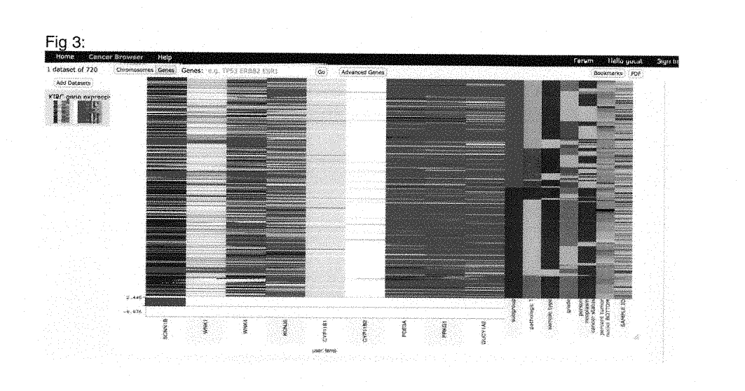

FIG. 2b. Cancer specific survival of subjects with prostate adenocarcinoma, with alterations in the said genes.

FIG. 3. Differential gene expression in patients with low (stage pT2 and lower) and high (stage pT3 and higher) stage renal cell carcinoma (clear cell type).

### DETAILED DESCRIPTION

Step 1: How did we identify the genes: 1) We looked up the list of high blood pressure genes published in various sources. Two such sources are: http://bws.iis.sinica.edu.tw/THOD/ and human-phenotype-ontology.github.io/. We use a statistical software to identify significant genes; 3) Top 5 or 10 or 20 genes; 4) Genes associated with high blood pressure, and closely linked to mTOR, PI3K, PTEN, and other known cancer genes; 5) While any of these methods described should not limit other ways to identify genes of interest in a particular patient, or a group of patients, as the genes attributed to causing high blood pressure is different in each individual, and so should the genes of high blood pressure related to causing cancer progression; 5) So, a highly expressed high blood pressure gene in an individual may be the target rather than a gene found to be most commonly expressed in patients with that particular caner. The genes of interest can be detected using microarray techniques known in the field.

How do we report the genes. The genes identified in a particular individual and the cancer risk profile may be generated into a report, so the patient may have this information. This report may also be detailed enough to provide necessary information to the treating physician.

In one embodiment, we selected the following set of genes from the list of high blood pressure genes: SCNN1B WNK1 WNK4 KCNJ5 CYP11B1 CYP11B2 PDE3A PRKG1 GUCY1A2. We then compared patients with stage 2 and lower cancer to stage 3 and higher cancer for difference in gene alterations, and gene expression.

These genes were picked from the list of high blood pressure genes. The gene names comply with the HUGO gene nomenclature committee guidelines.

In accordance with one embodiment, we used an online resource to explore the significance of these genes: http://www.cbioportal.org/. In this portal, we identified the subjects with renal cell carcinoma (clear cell type) of the TCGA, provisional data set comprising of 538 samples. We queried this website for the alterations in the said genes. We identified alterations in the genes as noted in FIG. 1a). We also noted that the cancer specific survival was significantly worse for subjects with alterations in the said genes. (FIG. 2a).

In accordance with one embodiment, we used an online resource: http://www.cbioportal.org/ to explore the significance of these genes. We identified the subjects with prostate adenocarcinoma of the TOGA, provisional data set comprising of 499 samples. We queried this website for the mutations, in the said genes. We identified alterations in the genes as noted in FIG. 1b). We also noted that the cancer specific survival was significantly worse for subjects with mutations in the said genes. (FIG. 2b).

In accordance with one embodiment, we used an online resource: https://genome-cancer.ucsc.edu/proj/site/hgHeatmap/ to explore the significance of these genes.

We identified the subjects with renal cell carcinoma (clear cell type) of the TOGA, provisional data set comprising of 538 samples. We queried this website for the mutations in the said genes. We then checked them for a statistically significant difference between patients with low (stage pT2 and lower) and high (stage pT3 and higher) stage renal cell carcinoma (clear cell type). (FIG. 3)

In one clinical scenario, a patient with Renal cell carcinoma, presented to the physician to be evaluated for the risk of his/her progression. Having identified the genes associated with worse prognosis in step 1, we used RT-PCR platform to identify the gene transcripts of the high blood pressure genes in the given patient. These genes may also be combined into a microarray as known in the field, to facilitate assessment of the patient sample for the gene alterations or gene expressions of interest. Some other techniques known in the field to identify the gene alterations include whole exome sequencing, and other gene sequencing technologies. These techniques of identifying the set of gene alterations, or gene expression in a patient are prior knowledge, and can be used effectively to identify the gene alterations or gene expression in a given patient. The test is performed on the biopsy of the cancer tissue, but could also be performed on the organ(s) harboring the cancer, blood, or other body fluids, circulating tumor cells, or stored tissue from the patient. The test could be performed serially in time to assess the changes in the genes of interest over time. The test sample if necessary is collected and stored in tubes that stabilize and prevent degradation of nucleotides or proteins of interest. The gene expressions are normalized against the expression levels of all RNA transcripts or their expression products in the tumor being evaluated, or a reference set of RNA transcripts or their products. If the gene alterations or the gene transcripts identified are among the genes associated with high risk for progression as identified in step 1, the patient can then be appropriately counseled on the appropriate treatment. Further, the treatment could be blood pressure lowering agents, agents that block the mutated gene(s) in that patient, or block the products of the gene(s). Further, serial measurement of the alterations in the gene or gene products could provide information related to the progression of the disease.

Similar method can be used to identify individuals potentially at higher risk of harboring high risk RCC. Additionally, potential treatments include blood pressure lowering agents and agents that block the by products of these genes, which can play a role in halting, reversing, or limiting the progression of the cancer. This is not limited to clear cell type renal cell carcinoma, or prostate cancer, and can be extrapolated to other tumor types as well. This is not limited to two groups of stage 2 and lower compared to stage 3 and higher. The comparison groups may include stage 1 to stage 2 and higher; stage 3 and lower compared to stage 4 and higher; or between any tumor classification types or between any tumor groups comparing lower to higher risk groups, as long as there is a statistically significant difference between the groups can be demonstrated. The difference in the genes can be used to identify individuals potentially at higher risk of harboring high risk RCC. Additionally, potential treatments include blood pressure lowering agents and agents that block the by products of these genes, which can play a role in halting, reversing, or limiting the progression of the cancer.

In other embodiments,

1. We create a training set (⅔rd of the cohort), and a validation set (⅓rd of the cohort). This division of dataset may be done by randomly picking patients into each of the sets randomly.

2. In the training set, we identify statistically different DNA mutations, methylation, differential gene expression, RNA and protein expression of genes between two groups. The two groups could be different tumor stages, age (eg:>70 years versus <=70 years), smokers vs non-smokers, alcoholics versus non-alcoholics, gender (male versus female), hormonal status (normal versus abnormal hormone levels or response), tumor versus controls, metastatic versus non-metastatic disease, or any other parameters to assess gene alterations and their differential expression. The groups could be more than two. The genes of interest can also be modified. Eg: if a patient has a certain gene altered, we could look for that gene in this model. Other methods of identifying genes of interest include any other well-known statistical methods in the field. One such method is top 5, or 10, or 20 altered genes could also be assessed by this method.

3. In the validation set, we confirm if the findings of the training step are true.

The entire steps 1 to 3 may be automated to perform approximately 1000 times, to ensure validity (that is, the identified mutations, methylation, RNA and protein expression; and agreement between the training and validation sets). There are several ways to perform statistical methods, and any such common knowledge methods can be used to identify the genes of interest. The comorbidity, gene, or gene products can then be evaluated for potential therapeutic targets, thereby achieving either a cure, or a delay in progression of cancer. In one embodiment, a blood pressure medication called angiotensin receptor blocker may be used to control high blood pressure. The same drug may be used to reduce the incidence of kidney cancer, and/or progression of kidney cancer. The resources such as: http://www.reactome.org and http://www.genome.jp/kegg/pathway.html provide us with necessary tools to explore drug targets, the method of which is within the realm of one skilled in the art.

In another embodiment, the gene alterations identified by statistical analysis could be ranked based on their close association with known cancer genes. The statistical analysis may also include multivariate analysis, with one more of the patient baseline characteristics data, gene alteration data, gene expression data being included in such multivariate analysis. Other common methods of statistical analysis known in the art may also be used. The links for some of the software available in the field are:

https://bioconductor.org/packages/devel/bioc/vignettes/TCGAbiolinks/inst/doc/tcgaBiolin ks.html

http://www.bioconductor.org

Conclusion, Ramifications, and Scope

Accordingly the reader will see that, according to one embodiment of the invention, we have provided a method to identify genes associated with medical comorbidities predicting worse cancer related outcomes, and a method of predicting cancer related risk of progression to an individual patient.

While the above description contains many specificities, these should not be construed as limitations on the scope of any embodiment, but as exemplifications of various embodiments thereof. Many other ramifications and variations are possible within the teachings of the various embodiments. For example, varying the number of patients could change the statistical significance, thus, one can find statistically significant genes merely by changing the number of patients, the baseline tumor characteristics of the patients, baseline comorbidity characteristics of the patients, etc. Thus the scope should be determined by the appended claims and their legal equivalents, and not by the examples given. It is important to note that high blood pressure, and other comorbidities are not a result of one or few gene alterations, but a complex interplay of multiple genes, in addition to environmental, economic, social, hormonal, and other factors. Similarly, cancer progression is also a complex interplay of multiple genes, in addition to environmental factors. Therefore, any analysis of the risk of cancer progression, and the results of such analysis needs to take this into consideration.

Definitions:

Unless defined otherwise, technical and scientific terms used herein have the same meaning as commonly understood by one of ordinary skill in the art to which this invention belongs. Singleton et al., Dictionary of Microbiology and Molecular Biology 2^{nd} ed, J. Wiley & Sons (New York N.Y. 1994), and, Advanced Organic Chemistry Reactions, Mechanisms and Structure 4^{th} ed., John Wiley & Sons (New York, N.Y. 1992), provide one skilled in the art with a general guide to many of the terms used in the present application. One skilled in the art will recognize many methods and materials similar or equivalent to those described herein, which could be used in the practice of the present invention. Indeed, the present invention is in no way limited to the methods and materials described. For purposes of the present invention, the following terms are defined below.

The term “microarray” refers to an ordered arrangement of hybridizable array elements, preferably polynucleotide probes, on a substrate.

The term “polynucleotide”, when used in singular or plural, generally refers to any polyribonucleotide or polydeoxy-ribonucleotide, which may be unmodified RNA or DNA or modified RNA or DNA. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical regions often is an oligonucleotide. The term “polynucleotide” specifically includes cDNAs. The term includes DNAs (including cDNAs) and RNAs that contain one or more modified bases. Thus, DNAs or RNAs with backbones modified for stability or for other reasons are “polynucleotides” as that term is intended herein.

The terms “differentially expressed gene”, “differential gene expression” and their synonyms, which are used interchangeably, refer to a gene whose expression is activated to a higher or lower level in a subject suffering from a disease, specifically cancer, such as kidney cancer, relative to its expression in a normal or control subject. The terms also include genes whose expression is activated to a higher or lower level at different stages of the same disease. It is also understood that a differentially expressed gene may be either activated or inhibited at the nucleic acid level or protein level, or may be subject to alternative splicing to result in a different polypeptide product. Such differences may be evidenced by a change in mRNA levels, surface expression, secretion or other partitioning of a polypeptide, for example. Differential gene expression may include a comparison of expression between two or more genes or their gene products, or a comparison of the ratios of the expression between two or more genes or their gene products, or even a comparison of two differently processed products of the same gene, which differ between normal subjects and subjects suffering form a disease, specifically cancer, or between various stages of the same disease. Differential expression includes both quantitative, as well as qualitative, differences in the temporal or cellular expression pattern in a gene or its expression products among, for example, normal and diseased cells, or among cells which have undergone different disease events or disease stages. For the purpose of this invention, “differential gene expression” is considered to be present when there is at least an about two-fold, preferably at least about four-fold, more preferably at least about six-fold, most preferably at least about ten-fold difference between the expression of a given gene in normal and disease subjects, or in various stages of disease development in a diseased subject.

The phrase “gene amplification” refers to a process by which multiple copies of a gene or gene fragment are formed in a particular cell or cell line. The duplicated region (a stretch of amplified DNA) is often referred to as “amplicon”. Usually, the amount of the messenger RNA (mRNA) produced, i.e., the level of gene expression, also increases in the proportion of the number of copies made of the particular gene expressed.

The term “diagnosis: is used herein to refer to the identification of a molecular or pathological state, disease or condition, such as the identification of a molecular subtype of head and neck cancer, colon cancer, or other type of cancer.

The term “prognosis” is used herein to refer to the prediction of the likelihood of cancer-attributable death or progression, including recurrence, metastatic spread, and drug resistance,,of a neoplastic disease, such as breast cancer.

The term “prediction” is used herein to refer to the likelihood that a patient will respond either favorably or unfavorably to a drug or set of drugs, and also the extent of those responses, or that a patient will survive, following surgical removal or the primary tumor and/or chemotherapy for a certain period of time without cancer recurrence. The predictive methods of the present invention can be used clinically to make treatment decisions by choosing the most appropriate treatment modalities for any particular patient. The predictive methods of the present invention are valuable tools in predicting if a patient is likely to respond favorably to a treatment regiment, such as surgical intervention, chemotherapy with a given drug or drug combination, and/or radiation therapy, or whether long-term survival of the patient, following surgery and/or termination of chemotherapy or other treatment modalities is likely.

The term “tumor” as used herein, refers to all neoplastic cell growth and proliferation, whether malignant or benign, and all pre-cancerous and cancerous cells and tissues.

The terms “cancer” and “cancerous” refer to or describe the physiological condition in mammals that is typically characterized by unregulated cell growth. Examples of cancer include but are not limited to, kidney cancer, prostate cancer, bladder cancer, breast cancer, lung cancer, colon cancer, hepatocellular cancer, gastric cancer, pancreatic cancer, cervical cancer, ovarian cancer, liver cancer, cancer of the urinary tract, thyroid cancer, melanoma and brain. Any other terms used in this application must be used in the context of use and interpretation as used by one skilled in the art.

The practice of the present invention will employ, unless otherwise indicated, conventional techniques of molecular biology (including recombinant techniques), microbiology, cell biology, and biochemistry, which are within the skill of the art. “Molecular Cloning” A Laboratory Manual”, 2^{nd} edition (Sambrook et al., 1989); “Parker & Barnes, Methods in Molecular Biology” (1999), Hod Biotechniques (1992), Trends in Genetics 1992. Genome Res. (2002) by Kent, W J. The method of using “Reactome” is available at http://www.reactome.org/userguide/Usersguide.html.

One skilled in the art will recognize many methods and materials similar or equivalent to those described here.

### REFERENCES:

1. Ljungberg, B., et al., The epidemiology of renal cell carcinoma. Eur Urol, 2011. 60(4): p. 615-21.

2. National Cholesterol Education Program Expert Panel on Detection, E. and A. Treatment of High Blood Cholesterol in, Third Report of the National Cholesterol Education Program (NCEP) Expert Panel on Detection, Evaluation, and Treatment of High Blood Cholesterol in Adults (Adult Treatment Panel III) final report: Circulation, 2002. 106 (25): p. 3143-421.

3. Frank, I., et al., Solid renal tumors: an analysis of pathological features related to tumor size. J Urol, 2003. 170(6 Pt 1): p. 2217-20.

4. Sahni, V. A. and S. G. Silverman, Imaging management of incidentally detected small renal masses. Semin Intervent Radiol, 2014. 31(1): p. 9-19.

5. Golub, T. R., et al., Molecular classification of cancer: class discovery and class prediction by gene expression monitoring. Science, 1999. 286(5439): p. 531-7.

6. McLaughlin, J. K., et al., International renal-cell cancer study. VIII. Role of diuretics, other anti-hypertensive medications and hypertension. Int J Cancer, 1995. 63(2): p. 216-21.

7. Li, Y. H., G. G. Zhang, and N. Wang, Systematic Characterization and Prediction of Human Hypertension Genes. Hypertension, 2017. 69(2): p. 349-355.

1. A method of identifying genes associated with poor cancer outcomes, comprising a group of patients with the said cancer, determining their comorbidities, determining the gene alterations associated with the said comorbidities, determining the gene expression level associated with the said comorbidities, normalizing said level against the expression level of a reference set of RNA transcripts, performing a statistical analysis, generating a plurality of groups of patients with varying cancer risk, creating the list of statistically significant genes wherein the expression level of said genes can determine cancer specific survival.

2. The method of claim 1 wherein the cancer outcomes may comprise worse cancer stage, with treatment or without treatment.

3. The method of claim 2 wherein the treatment may include and not limited to one more of the following: surgery, radiation, chemotherapy, watchful waiting, active surveillance, immunotherapy, thermotherapy, embolization, cryotherapy.

4. The method of claim 1 wherein the gene interactions with cancer genes may be identified using software wherein a suitable drug targeting the interaction or targeting the cancer gene may be used.

5. The method of claim 1 wherein the comorbidities include high blood pressure.

6. The method of claim 1 wherein the groups may comprise cancer in different stages grouped into two or more groups.

7. The method of claim 1 wherein the groups may comprise different ages of two or more groups.

8. The method of claim 1 wherein the groups may comprise different genders or hormonal status.

9. The method of claim 1 wherein the groups may comprise different alcohol intake of two or more groups.

10. The method of claim 1 wherein the groups may comprise different smoking status of two or more groups.

11. The method of claim 1 wherein the genes include one or more of the genes listed in either table 1 or table 2.

12. The method of claim 1 wherein the gene alteration and gene expression is quantified.

13. The method of claim 1 wherein the genes identified as significant are further assessed for their relevance to oncogenes.

14. The method of claim 1 wherein the genes and their products may be blocked by using a suitable drug thereby achieving either a cure, or a delay in progression of cancer.

15. A method of identifying the risk of an individual cancer patient, comprising the gene formation obtained for the said patient for the said cancer from claim 1, obtaining baseline patient data such as age, gender, hormonal status, tumor stage, alcohol intake, smoking status, treatment received for the said cancer, obtaining tissue for gene analysis from the said patient, performing gene analysis in the said tissue, obtaining gene expression of the said cancer, comparing to the results obtained in claim 1, performing a statistical analysis, creating a report of the said analysis, wherein the patient and the physician can thereby assess best treatment strategy.

16. The method of claim 15 wherein the patient may have comorbidities of high blood pressure.

17. The method of claim 15 wherein the quantitative determination of the gene alteration and gene expression is predictive of cancer survival.

18. The method of claim 15 wherein the sample for gene analysis is obtained from the patient at one more time points in the course of their clinical evaluation for the tumor.

19. The method of claim 15 wherein the gene alterations, gene expression, and/or protein expression may be obtained using commercially available methods, kits comprising plurality of gene probes, or using previously issued reports.

20. The method of claim 15 wherein the genes and their products may be blocked by using a suitable drug, thereby achieving either a cure, or a delay in progression of cancer.
